(19) United States
(12) Patent Application Publication
(10) Pub. No.: US 2015/0320522 A1
(43) Pub. Date: Nov. 12, 2015

(54) ANGULAR CONNECTOR/MILLING CUTTER

(71) Applicant: Jeder GmbH, A-1190 Wien (AT)

(72) Inventor: KLAUS EDER, Perchtoldsdorf (AT)

(21) Appl. No.: 14/411,459

(22) PCT Filed: Jun. 21, 2013
(86) PCT No.: PCT/AT2013/050123

§ 371 Date: Dec. 26, 2014

(30) Foreign Application Priority Data
Jun. 28, 2012 (AT) A 728/2012

(51) Int. Cl. A61C 8/00; A61C 1/00; A61C 1/14; A61B 17/16; A61C 1/12

(52) U.S. Cl. CPC A61C 8/0092; A61B 17/1673; A61C 1/12; A61C 1/141; A61C 1/0061; A61B 2017/00473

(57) ABSTRACT

A device for the perforating extension of a blind bore introduced into a jawbone, includes a hollow body defining a hollow space and having a distal working opening and an inlet opening opposite the working opening, and being sealingly insertable into the blind bore, a working tool connected with the hollow body so as to form a common unit with the hollow body, a shaft arranged on the working tool and having a distal tool head for working on the jawbone, said shaft being insertable into the hollow space through the inlet opening, said distal tool head being at least partially guidable through the working opening for working on the jawbone, a connection fluidly connected to the hollow body for establishing an internal pressure in the hollow, and an adjustment mechanism provided in or on the device for axial movement or linear forward feed of the shaft in the hollow space along a longitudinal axis of the shaft and the hollow space

The invention relates to a device for perforating extension of a blind bore introduced in a heart tissue in particular into a jawbone according to the preamble of claim 1.

Such an extension of the bone bore is for example required in the field of dental surgery when performing a procedure, which is referred to as sinus lift.

This sinus lift is a surgery in which the mucosa of the maxillary sinus or sinus membrane or Schneider's membrane is partially detached and lifted in order to create a space between the bone and the mucosa of the maxillary sinus. Into the created hollow space for example a synthetic bone replacement material is introduced. This material is intended to convert into bone within 6 months to ensure a durable foundation for an implant.

The method of the open or classical sinus lift however requires a folding open of the remaining bone plate and is relatively invasive. A more recent method is the so-called “crestal” sinus lift which does not require a folding open but a peroration of the bone plate.

Even though very advanced methods have already been developed in order to carefully and sufficiently detach the mucosa of the maxillary sinus through a small bore, which in most cases has a diameter of about 4 mm, the moment at which the jawbone is perforated remains a critical moment which requires great experience and special skill, even when taking all necessary care there remains a residual risk that the fragile mucosa of the maxillary sinus is damaged during perforation of the bone plate.

In order to render the sinus lift procedure more safe, means are desirable which facilitate this perforating extension of the jawbone bore so as to decrease the risk of causing damage to the fragile mucosa of the maxillary sinus behind the jawbone.

Such an advantageous means is for example known from WO 2010/048648 A1, in which a device is described that has a tubular body with a distal working opening and an entry opposite the working opening, which is sealed by a sealing element which is traversed by a shaft of a working tool, for example a milling cutter. The tubular body is sealingly inserted into a blind bore introduced beforehand into the jawbone, wherein the distal working opening abuts the end of the blind bore thereby sealingly closing the inner space to the most degree. The working medium present in the inner space of the tubular body, preferably a NaCl solution, can be pressurized via the connection, for example by means of a syringe connected with the connection. With the working tool, which can be separately controlled from the outside, the bone plate remaining between the blind bore and the maxillary sinus is now milled away in the region of the working opening. At the moment at which the head of the working tool perforates the bone and enters the region below the sinus membrane the overpressure in the interior of the tubular body causes the working media to enter through the free opening and to push the sinus membrane situated behind the free opening away from the bone and thus out of the working area of the working tool and the zone of danger. The outflow of the working medium causes a decrease in pressure, which indicates the perforation of the bone and also prevents an excessive ballooning of the sinus membrane. Such a device can be reliably operated and excellent surgical results can be achieved with such a device with a minimal risk to the sinus membrane.

A further advantageous device working according to the same principle is known from patent AT 501.402.

A certain disadvantage in these devices is however that two hands are required for their operation. The reason for this is that these devices are formed by two independently operable and not interconnected components namely the tubular body and the working tool.

The dentist holds the tubular body with one hand and has to simultaneously exert a certain pressure in order to sealingly insert the tubular body into the blind bore and to position it on the mucosa because otherwise it would not be possible to build up a sufficient pressure in the interior of the tubular body. With the other hand the dentist has to hold the working tool, usually a drill, and has to precisely guide the working tool in order to remove the remaining bone plate. Hereby the dentist must position the shaft or the drill head correctly and on the other hand exert the right pressure required for work on and removal of the bone plate. As mentioned especially the removal is very difficult and requires a steady hand; excessive pressure or a jerking advance of the drill head after drilling through the bone plate can easily result in rupture of the sinus membrane.

Especially the fact that the dentist has to use both hands and in addition has to perform different movements with the two hands, complicates the operation of these known devices and increases the risk.

It is therefore an object of the invention to further develop the above mentioned devices while retaining their advantageous functionality so that the operation of the device is made easier while maintaining operational safety.

This object is solved by the characterizing features of claim 1.

The fact that the working tool and the hollow body are interconnected into a common constructive unit, makes it possible for a dentist to hold and operate the entire device with only one hand. This by itself would not be sufficient because a mere constructive connection of these two components would no longer ensure the functionality of the device, i.e., the safe removal of the remaining bone plate.

This is because at the same time an adjustment mechanism constructively formed or arranged in or on the device according to the invention has to be provided for axial guidance and back and forth movement and for linear forward feed of the shaft in the hollow space along the longitudinal axis of the shaft or the hollow space. Only this makes it possible for the dentist to insert the hollow body into the blind bore with only one hand and at the same time to exert the corresponding pressure and to build up pressure in the hollow body. This also enables controlling the forward feed of the shaft or the drill head and removing the remaining bone plate in a targeted manner. When the drill perforates the bone plate the inner pressure immediately pushes the sinus membrane out of the zone of danger, at the same time the pressure decreases and the dentist stops the forward feed.

The other hand of dentist remains free during this period for further activities. Thus the dentist can for example manually vary the pressure in the interior with the syringe with the other hand, and adjust the rotation or turning speed of the drill or the forward feed. However other instruments for example a Langenbeck can also be held and positioned with the free hand.

The features of the dependent claims describe further advantageous embodiments of the device according to the invention.

Thus according to an advantageous embodiment it is provided that the working tool and the hollow body are interconnected at a fixed predetermined distance to each other, preferably at a fixed position relative to each other and only the shaft is movable relative to the working tool linearly along the longitudinal axis of the shaft and the hollow body. The adjustment mechanism for the linear movement of the shaft is preferably exclusively arranged in the working tool, preferably in the interior of the angular connector head. The adjustment mechanism is a durably constructed instrument in which all essential movable parts are arranged in the interior so as to be protected. Also this device is dimensioned relatively small and can therefore be advantageously used in regions of the oral cavity that are hard to access.

Advantageously two separate drives are provided, a first drive which exclusively causes rotation of the shaft 5 and a second drive which exclusively causes the linear movement of the shaft 5 in the direction of the arrow. Depending on the circumstances both drives are arranged in the interior of the angular connector head. In this connection it is advantageous when the working tool and the hollow body are interconnected via a reversibly detachable connection, in particular a screw connection or a bayonet connection. In this way the interior components, in particular the adjustment mechanism and the guide element, can be easily accessed.

An alternative embodiment provides that the shaft has a fixed distance to the working tool or is not movable in linear direction relative to the working tool along the longitudinal axis of the hollow space. On the other hand the working tool and the hollow body are interconnected so that their distance to each other is variable, wherein the distance between the working tool and the hollow body is exclusively adjustable along the longitudinal axis of the hollow body. The distance between the working tool and the hollow body is hereby adjusted via mechanical adjustment means outside the angular connector head. Such an embodiment may be somewhat larger dimensioned, however does not require an interior miniaturized drive technology.

For ensuring an exact parallel displacement it is advantageous when the working tool and the hollow body are interconnected via a straight threaded bolt, which is preferably oriented parallel to the longitudinal axis of the shaft and the hollow space so as to enable adjustment of a distance between the working tool and the hollow, wherein the threaded bolt is preferably rotatable via an external drive thereby enabling adjustment of the distance. Via the threaded bolt the forward feed of the shaft can at the same time be actuated in a controlled manner.

A further advantageous possibility for ensuring the parallel displacement of the components relative to each other is characterized in that at least one, optionally two guide pin(s) protrude(s) from projections extending from the hollow body, which guide pin(s) are/is oriented parallel to the longitudinal axis of the shaft and the hollow space and which traverse(s) a corresponding recess of the working tool and is/are guided therein.

Hereby a spacer is advantageously arranged between the working tool and the hollow body whose volume and/or thickness or height can be adjusted.

Advantageously the spacer is a hollow body, whose volume changes through supply or discharge of a fluid, in particular a balloon body, whose size can be increased by water.

In this connection it is particularly advantageous when the spacer is a ring, which surrounds the shaft and rests against the working tool as well as the hollow body in the region of the inlet opening. Such an embodiment is constructively and mechanically configured very simple. Especially no real drive is required for the displacement of the components relative to each other, rather this displacement can be effected by manually filling or emptying the spacer via a syringe.

In praxis, drills or milling cutters with rotating shafts have proven useful and it is therefore advantageous when the working tool is an angular connector with a shaft extending from its angular connector head and rotating about its longitudinal axis, preferably with a working head constructed as a milling cutter or drill head. The device according to the invention therefore enables use of such drills in spite of the fact that especially drills and milling cutters pose the highest risk of injury to the sinus membrane.

The device advantageously only requires a single handle via which the working tool and the hollow body can be simultaneously grasped and operated with only one hand.

In praxis it has proven sufficient when the travel by which the shaft is movable is maximally 1 cm, preferably about 0.6 cm. However such small travels are nevertheless sufficient to cause damage to the sinus membrane.

For additionally increasing the sealing of the system and for increasing the inner pressure it is possible that the inlet opening is closed with a sealing element which enables at least a linear adjustment of the shaft.

A further advantageous embodiment provides that a guide element is provided which is insertable into the inlet opening with a precision fit, wherein the inlet opening is closed pressure tight and essentially fluid tight by the guide element, wherein the guide element has a continuous recess through which the shaft can be guided and inserted into the hollow body and wherein the guide element is optionally a connection for a working medium for establishing an internal pressure in the pressure chamber or the hollow body.

The guide element is optionally fastened on the hollow body so as to be detachable from the hollow body in a destruction free manner. In this way it is possible that the components, which are easily contaminated during operation, can be easily replaced. Such a guide element is for example described in AT 510.402.

It is advantageous when the shaft is optionally supported and guided in the recess in a pressure tight and essentially fluid tight manner, and a pressure of at least 1.5 bar, preferably at least 2.5 bar, can be established in the pressure chamber and at the same time at least one forward, drive and/or control movement of the shaft, for example a rotation, a circulating or tumbling movement and/or an axial forward feed of the shaft is ensured.

Preferably the shaft can only or exclusively perform an axial or linear movement according to the arrow.

A very simple and automated solution provides that the rotation of the shaft is coupled with the linear forward feed of the shaft via a common drive. With this the number of control buttons on the working tool can be reduced for the dentist.

Further advantages and embodiments of the invention will become apparent from the description of the included drawings.

The invention is schematically shown by way of different exemplary embodiments in the drawings and is exemplary described in the following with reference to the drawings.

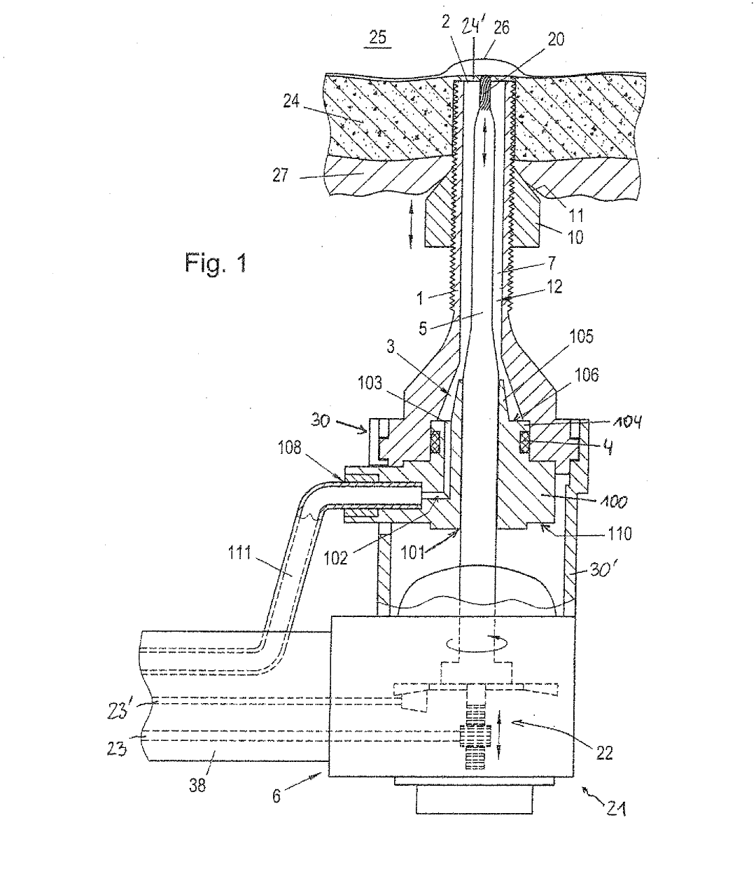

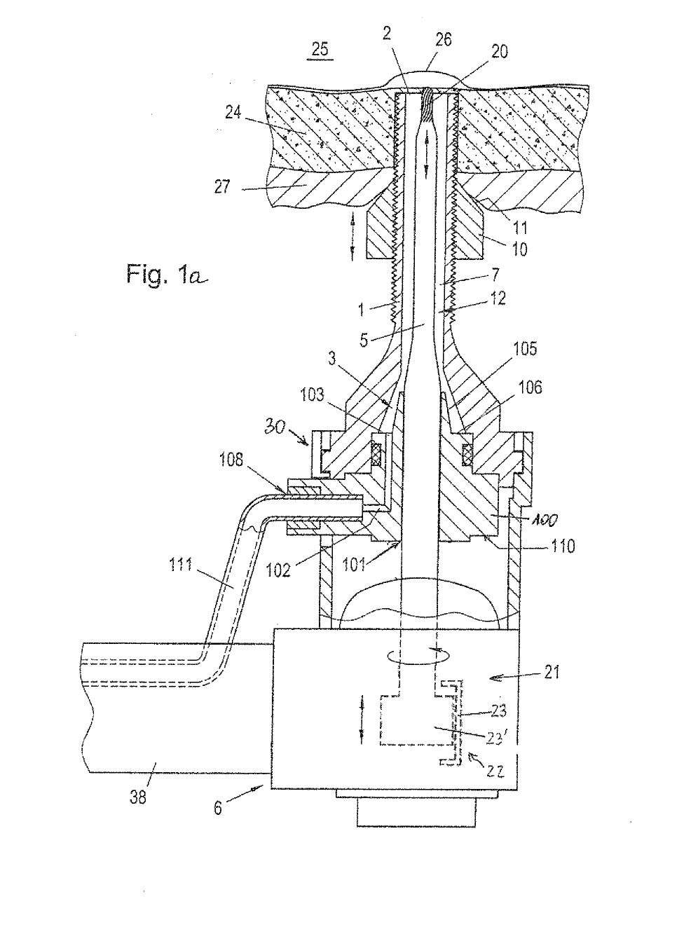

FIG. 1 shows a first embodiment of the invention in a cross section.

FIG. 1a shows a modification of the first embodiment in a cross section.

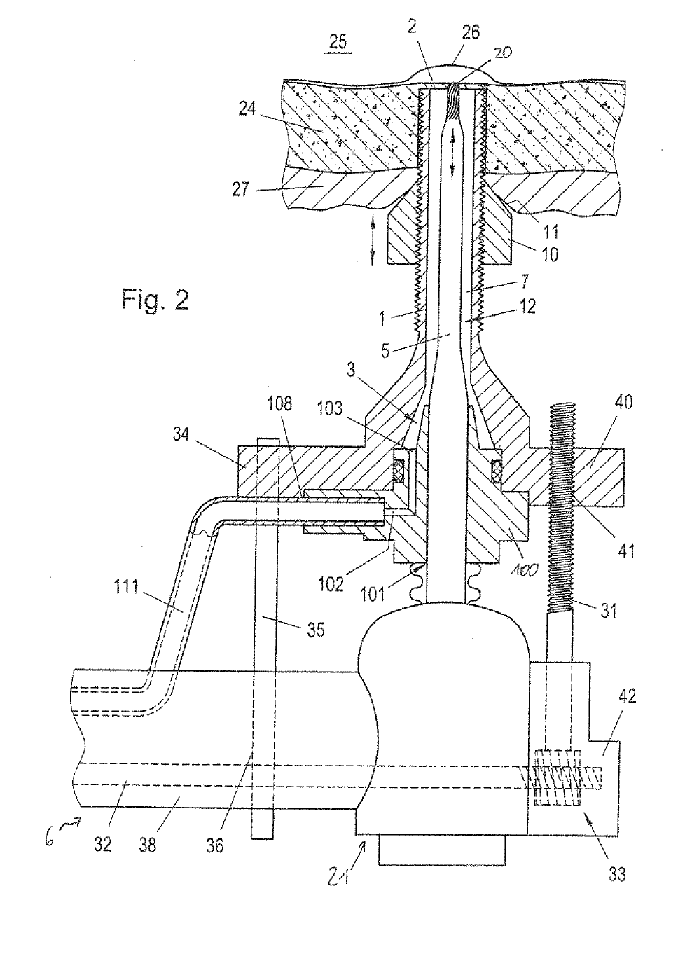

FIG. 2 shows a second embodiment of the invention in cross section.

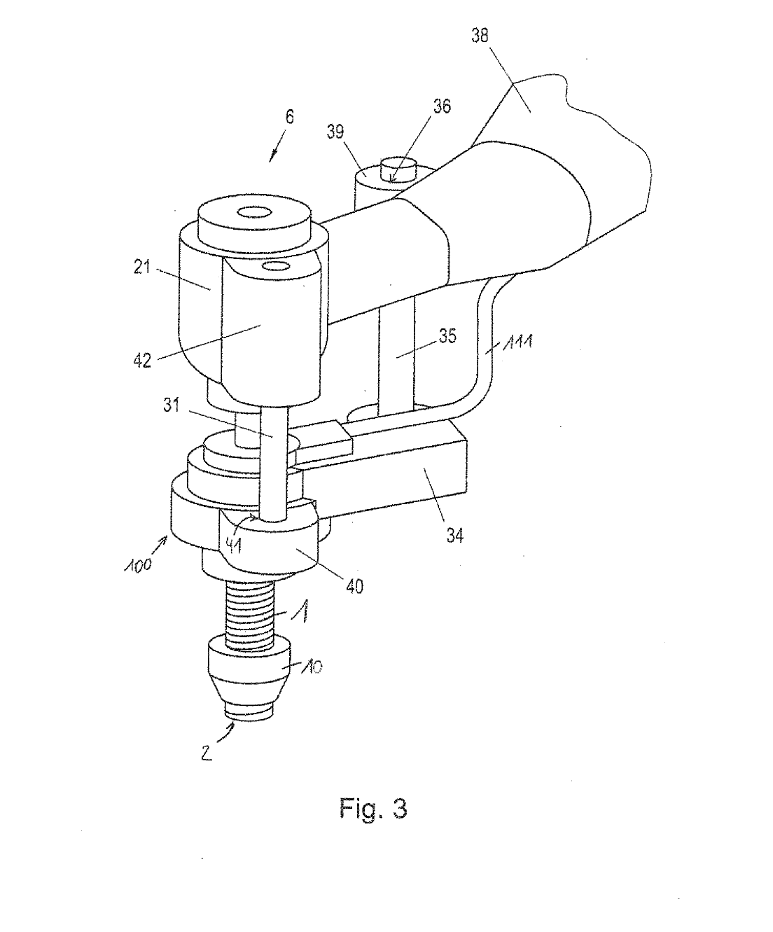

FIG. 3 shows a perspective view of a part of the device according to FIG. 2.

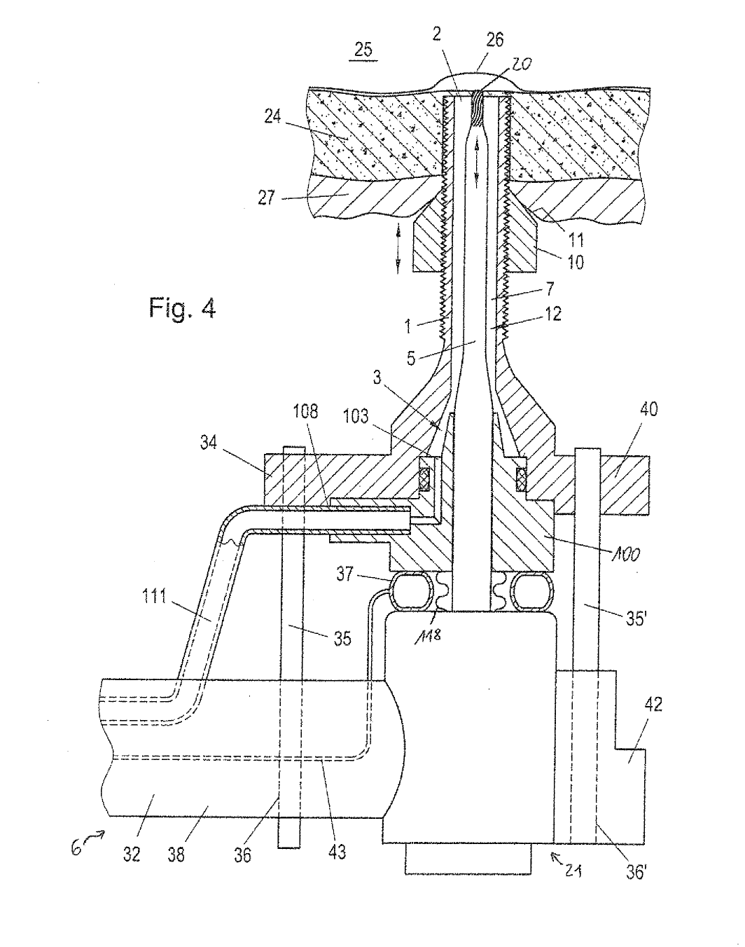

FIG. 4 shows a third embodiment of the invention in cross section.

The general construction and the functionality of the devices shown in the FIGS. 1 to 4, and in particular their correct use on the patient, are already described in detail in WO 2010/048648 A1. The construction of the guide element 100 and analogous devices having such a guide element 100, are described In AT 510 402.

The exchangeable guide element 100 serves beside the sealing function also to significantly increase the contamination safety or decrease of the infection risk of the device and is not strictly required for the pure functionality of the device, i.e., for the safe working so as to avoid ruptures of the sinus membrane. The device can also be used without this guide element 100 as described for example in WO 2010/048648 A1 so long as the tightness in the pressure chamber 7 or the hollow space 12 is ensured or the shaft 5 is sufficiently sealed through other measures. In the following representations of the embodiments of FIGS. 1 to 4, a guide element 100 is nevertheless provided. The corresponding features and reference signs are consistent in the Figures.

The guide element 100 is formed by a sterilizable polymer, is constructed one-piece or one-part produced by means of injection molding process. In a disc-shaped head region 110 of the guide element 100 a central cylindrical recess 101 is formed, which completely traverses the guide element 100. Through this recess 101 the shaft 5 of a working tool 6 for example a milling cutter is guided.

Adjoining the head region 110 is a connection 108 in the form of a socket for fastening a tube 111. The connection 108 extends radially outwardly from the center point of the recess 101 and the central longitudinal axis of the connection 108 is situated in the plane of the disc-shaped head region 110 which is oriented parallel to the surface.

The bottom surface 106 of the guide element 100, which during operation faces the bone 24, is configured smooth and planar which ensures a good slidability and pivotability. In the head region 110 a cylindrical projection 104 is formed which is surrounded by a sealing element 4 in the form of an O-ring, which is partially received in a groove. With this the guide element 100 is inserted in the device with an exact fit.

On the projection 104 a frustoconical sleeve 105 is molded which tapers upwardly. The greatest diameter on the basis of the sleeve 105 is smaller than the diameter of the cylindrical projection 104 by about 30 to 40%.

The cylindrical projection 104 as well as the sleeve 105 and also the head region 110 are arranged concentric about the central longitudinal axis of the recess 101 and are centrally traversed by the recess 101.

Extending from the connection 108, is a channel 102, which is closed from all sides and which is entirely situated in the interior of the guide element 1012, and which connects the connection 108 with the outlet opening 103. This outlet opening 103 leads out of the bottom surface of the cylindrical projection 104 adjacent the sleeve 105 and enables introduction of the fluid working or pressure medium, in most cases physiological saline solution, into the inlet opening 3 of the device. The channel 102 is separated from the recess 101 along its entire extent. The channel 102 has a subsection proximate to the connection 108 with an initial greater diameter, which is essentially oriented perpendicularly and radially to the central longitudinal axis of the recess 101. The cross section of the channel 102 then becomes smaller and branches off at a right angle. This second adjoining subsection extends essentially parallel to the central longitudinal axis of the recess 101. In this way the channel 102 can be configured constructively simple in the one-piece guide element 100, for example by two retroactively introduced bores.

FIGS. 1, 1a, 2 and 4 each show a cross section through different embodiments of the device in operation during a sinus lift, i.e., at the critical time point when the working head 20 perforates the bone plate 24′ of the jawbone 24 of the upper jaw.

The working tool 6 is in most cases a conventional angular connector or a drill with a front angular connector head 21 with a shaft 5 inserted therein and a tool head, in particular a drill head 20, arranged at the end of the shaft 5 for working on the jawbone 24.

The tubular hollow body 1 has a substantially cylindrical inner hollow space 12, a distal working opening 2 and an inlet opening 3 opposite this working opening 2. Through this inlet opening 3 the pressure medium as well as the shaft 5 of the working tool 6 are introduced into the hollow space 12 of the hollow body 1. The hollow body 1 is substantially constructed as the tubular body described in WO 2010/048648 A1.

In order to form a pressure chamber in the interior of the hollow body 1, the tubular hollow space 12 of the hollow body 1 as well as the shaft 5 of the milling cutter 6 have to be sealed in the region of the inlet opening 3. The thus generated pressure chamber 7 and the pressure establish therein ensures that the sinus membrane 26 is timely pushed out of the zone of danger when perforating the bone plate 24′.

The forming pressure chamber 7 can either be situated entirely in the interior of the hollow body 1 when the working opening 2 sealingly ends flush with the bottom of the blind bore, the pressure chamber 7 can however also extend into a region outside of the actual hollow body 1 which is delimited by the walling of the blind bore and the conical sealing unit or the flange 10, 11. During use both has a sealing effect and both sealings complement each other in order to maintain the system as pressure tight and fluid tight as possible.

The inlet opening 3 on the other end of the pressure chamber 7 is sealing closed by the exact fit and form fittingly inserted guide element 100. The shaft 5 is inserted and traversed the recess 12-1. The tube 111 is connected on the connection 108 and thus enabling supply of pressure medium into the interior of the hollow body 1. The tube 111 can lead to a syringe or a manual or automatic pressure control unit with which the pressure can be introduced and controlled.

As in the conventional crestal sinus lift, using the device according to the invention also involves a prior procedure in which first a blind bore is introduced into the jawbone 24 from the jaw crest, wherein an approximately 1 mm thick bone plate 24′ remains between the end of the blind bore and the maxillary sinus 25. This is necessary in order to prevent damage to the sinus membrane 26, which rests against the jawbone 24, in the maxillary sinus 25.

The hollow body 1 is then sealingly inserted into the prepared blind bore until the working opening 2 abuts the bone plate 24′. The hollow body 1 stands still during the treatment is fixed in position and can even be screwed into the blind bore.

For improving the sealing effect, the flange 10 is shifted on the tubular body 1 toward the jawbone 24 so that the conical sealing socket 11 arranged on the flange 10 on the outer border of the blind bore is tightly pushed against the oral mucosa 27 thereby additionally sealing the blind bore. Optionally a rubber dam can also be used. In the interior of the pressure chamber 7 a hydrostatic pressure of for example 0.5 to 3 bar can thus form as a further consequence.

The working medium in the pressure chamber 7 serves at the same time for discharging heat generated during the milling and acts as lubricant for the rotating shaft 5. Small amounts of working medium that may escape along the rotating shaft 5 do not pose a problem for the functionality of the device, because the pressure of the working medium in the pressure chamber 7 can be maintained via the connection 108. Good sealing properties are nevertheless very advantageous because the pressure drop at the moment of perforation of the bone disc can be better recognized in the case of a tight pressure chamber 7.

The shaft 5 of the working tool 6 is introduced into the interior of the hollow body 1 through the recess 101 and the inlet opening 3. In addition the outermost tip of the tool head or drill head 20 of the milling cutter 6 or the shaft 5 is curved to a relatively strong degree or is configured punctifrom in order to ensure a most punctiform and small-surface penetration through the bone plate 24′.

The shaft 5 completely traverses the recess 101, the inlet opening 3 and the inner hollow space 12 of the hollow body 1 and exits during operation through the outlet opening 2 so that the remaining bone plate 24′ can be worked on. The distal part of the shaft 5 with small diameter can be guided through the recess 101 relatively easily the rear region with greater diameter then sits in the recess 101 with an accurate fit. The shaft 5 thus rests sealingly against the inner surface of the recess 101 or is additionally also sealed, guided and supported in the region of the cylindrical projection 104 and the sleeve 105. In the interior of the recess 101 an additional sealing, for example an O-ring, could also be provided which, however, is not the case in the present exemplary embodiment.

The shaft 5 is shiftable in the hollow space 12 according to the arrow along its longitudinal axis axially upwardly and downwardly or linearly back and forth, which is in particular required in the case of rotating milling cutter 6 in order to achieve the required forward feed for milling through the remaining bone plate 24′ in the jawbone 24.

When a working tool 6 is used with a shaft 5 that rotates about its own axis, the shaft still has to be able to rotate in spite of the guiding and the linear movability of the shaft 5. Still, the shaft is supported fluid tight and pressure tight in particular in the region of the sleeve 105 and a sufficient pressure can be built up in the pressure chamber 7 when the hollow body 1 is sealingly inserted in the blind bore.

Via the connection 108 the working medium, for example a physiological saline solution, can be introduced through the channel 102 and the outlet opening 103 into the inlet opening 3. Because the outer walling of the sleeve 105 is spaced apart from the inner walling oft the funnel-shaped inlet opening 3, the rinsing medium flows outside along the sleeve 105 and along the shaft 5 into the inner hollow space 12 or the pressure chamber 7.

During operation the milling cutter 6 or the shaft 5 is shifted further and further downwardly so that the tool head 20 exits on the lower end through the working opening 2 and contacts the bottom plate 24′ wherein the forward feed of the shaft 56 or the milling cutter 6 is very slow, for example 1 mm/min.

As soon as the bottom plate 24′ is minimally perforated at one site, which is supported by the punctiform configuration of the drill or milling cutter head 20, the pressurized working medium immediately flows through this minimal opening and the sinus membrane 26 is immediately pushed out of the zone of danger away from the drill head 20 and is slightly detached from the jawbone 24 even before the drill head 20 can completely penetrate through the opening. This corresponds to the situation shown in the Figures.

The milling cutter 6 is then turned off and as known from WO 2010/048648 A1, the sinus membrane 26 may be further detached in order to increase the space for the bone replacement material, for example by additional supply of pressurized medium and optionally applying vibrations. The shaft 5 can hereby initially remain in the recess 101 as sealing element. As an alternative the shaft 5 can also be removed and the recess 101 be sealed by inserting a plug. This enables further exerting pressure or maintaining the pressure constant. Depending on the circumstances vibrations, for example ultrasound vibrations, may also be introduced into the medium in order to facilitate further detachment of the sinus membrane 26.

In the embodiment according to FIG. 1 and FIG. 1a the angular connector head 21 of the drill 6 is connected with the hollow body 1 in a rigid, still positionally fixed and most of all constant predetermined fixed distance. This connection is accomplished by a detachable but rigid bayonet connection 30. The end region of the hollow body 1 which faces away from the jawbone 24, is hereby inserted into a cylindrical sheath 30′ which protrudes from the angular connector head 21, and latchingly engaged with the bayonet connection 30 and positionally fixed by a rotation about the longitudinal axis by about 10° to 30°.

The shaft 5 however has to be always supported so as to be linearly adjustable, otherwise removal of the remaining bone plate 24′ would not be possible by the forward feed of the tool head 20. For this purpose means 22 for linear adjustment of the shaft 5 or an adjustment mechanism 22 for movement of the shaft 5 are provided, wherein this adjustment mechanism 2 is essentially at least partially arranged in the interior of the angular connector head 21.

This adjustment mechanism 22 can be configured in different ways. The adjustment mechanism 22 usually includes a separate drive 23 for the shaft 5, which exclusively serves for the linear back and forth movement of the shaft 5 along the central longitudinal axis of the shaft 5 or the hollow space 12. At the same time the shaft 5 is driven rotatingly and is rotated during operation of a drill or milling cutter with high speed about its longitudinal axis. For this a further separate drive 23′ is often provided.

A first possibility according to FIG. 1 is that two separate drives are provided, i.e., a first drive 23′ exclusively for the rotation of the shaft 5 and a second drive 23 which exclusively displaces the shaft 5 linearly relative to the angular connector head 21. The two drives 23, 23′ are situated in the handle outside the angular connector head 21 and are controllable separate from each other, for example via operating elements arranged on the working tool 6 or via a foot pedal. In the angular connector head 21 corresponding mechanical couplings and gears as well as sprockets are provided in order to convert the movement of the drives 23, 23′ into the rotating and linear movement of the shaft 5.

A second possibility according to FIG. 1a is to provide two separate drives, i.e., a first drive 23′ exclusively for the rotation of the shaft 5 and a second drive 23, which displaces the shaft 5 together with the first drive 23′ exclusively linearly in the direction of the arrow along the longitudinal axis. The two drives 23, 23′ are arranged in the interior of the angular connector head 21 and can be controlled separate from each other.

As an alternative, it is also possible to couple the movements of the two drives 23, 23′, wherein an increase of the rotational speed of the shaft 5 simultaneously causes a slow forward feed in the direction of the jawbone 24 and a decrease in the drilling speed may cause a stop or a backward movement of the working head 20.

An alternative embodiment of the device according to the invention is described in FIGS. 2 and 3. The working tool 6 in this case is principally a conventional drill or a conventional angular connector with an angular connector head 21 in which a shaft 5 is fixedly inserted and can be rotated about its own axis at high speed. The shaft 5 in this case is thus not adjustable in linear direction along the central longitudinal axis of the hollow space 12. However, in the present embodiment, the working tool 6 or the angular connector head 21 and the hollow body 1 are connected with each other so as to enable adjusting the distance between each other. The two components 1, 21 are hereby exclusively movable back and forth linearly along the direction of the longitudinal axis of the shaft 5.

In the embodiment according to FIG. 2 this is accomplished by providing a straight threaded bolt 31 with an outer threading. This threaded bolt 31 is guided on one of its sides in a threaded channel 41 with inner threading in a partially cylindrical element 40 arranged on the hollow body 1. This element 40 is formed on a front lateral side of the hollow body 1 as can be seen in FIG. 3.

On its opposite side the threaded bolt 31 is also guided or supported in an element 432 protruding from the angular connector head 21. This element 42 is also partially cylindrical and like the element 40 is also formed on a front left hand side on the angular connector head 21. In the element 42 a pivot mechanism 33 for a not shown drive is located, which causes slow rotation of the threaded bolt 31 via a, optionally flexible, drive shaft 32 with about 2 to 3 revolutions per second. This device can be for example a foot pedal-operated electric motor. This ensures that during operation the entire working tool 6 and with this also the shaft 5 moves forward in the direction of the bone 24 and thereby the tool head 20 can work on the remaining bone plate 24′. The linear adjustability is about 0.6 cm.

On the opposite side of the handle 38 a straight guide pin 35 is arranged which starting from a socket 34 of the hollow body 1 is oriented parallel to the threaded bolt 31. The guide pin 35 has no threading and is smooth and passes through a recess 36 arranged on the working tool 6. The recess 36 is formed in a partially cylindrical socket 39 of the working tool 6 arranged laterally on the sole handle 38. The guide pin 35 is situated on the side of the handle 38, which is opposite to the threaded bolt 31. The guide pin 35 can be shifted with accurate fit in the corresponding recess 36, a lateral play or a tumbling movement is not possible. The guide pin 35 correspondingly ensures an exact parallel displacement of the components 1 and 21 to each other.

A further alternative embodiment of the invention is shown in FIG. 4. The general functional principle is analogous to the embodiment according to FIGS. 2 and 3, however no threaded bolt 31 is provided. Instead of the threaded bolt 31 a second straight guide pin 35′ without treading is provided. This further guide pin 35′ extends analogously from a partially cylindrical element 40 of the hollow body 1 or is fixed thereon and leads parallel to the first guide pin 35 in the direction of the working tool 6. On the working tool, namely on the angular connector head 21, a partially cylindrical element 42 with a corresponding recess 36′ is provided, wherein the recess 36′ is arranged and oriented so as to be aligned with a further guide pin 35′ and the guide pin 35′ is guided therein with an accurate fit and linearly displaceable. In this way an exact parallel displacement of the angular connector head 21 which carries the shaft 5 and the hollow body 1.

In this embodiment the adjustment of the distance between these two components 1 and 21, usually about 0.6 mm, is not caused by a drive but by a spacer 37. The spacer is a hollow body in the form of a ring or tire, whose volume can be adjusted and which is arranged about the shaft 5. The ring-shaped spacer 37 is situated between the hollow body 1 or the guide element 100 and the angular connector head 21 and is in contact with both. Via a separate tube 43 a fluid can be supplied to or removed from the spacer 37, thereby changing the volume of the spacer. Supply of fluid expands the spacer 37 and increases its thickness or height, causing the spacer to push the angular connector head 21 and with this the shaft 5 away from the bone 24. When fluid is removed in a controlled manner from the spacer 37, its height decreases and the angular connector head 21 and with this the shaft 5 are displaced linearly forwardly in the direction of the bone 24. The dentist can thereby accurately control the forward feed of the shaft 5 during operation.

Between the guide element 100 and the working tool 6 an elastic bellow 118 can additionally be arranged as is the case in FIGS. 2 and 4.

It is also possible to combine the embodiments according to FIG. 1, 1a with the embodiments according to FIGS. 2, 3 and 4, for example by additionally integrating the adjustment mechanism 22 or the drives 23, 23′ in the working tools 6 of the embodiments according to FIGS. 2, 3 and 4.

In this connection it is also possible to fix the threaded bolt 31 and/or the guide pins 35, 35′ for example by screw nuts reversibly and temporarily in a defined position.

What is claimed is:

1.-16. (canceled)

17. A device for the perforating extension of a blind bore introduced into a hard tissue, in particular into a jawbone, comprising:
a hollow body defining a hollow space and having a distal working opening and an inlet opening opposite the working opening, said hollow body being sealingly insertable into the blind bore, or comprising means for sealing insertion of the hollow body into the blind bore;
a working tool connected with the hollow body so as to form a common unit with the hollow body;
a shaft arranged on the working tool and having a distal tool head arranged on an end of the shaft for working on the jawbone, said shaft being insertable into the hollow space through the inlet opening, said distal tool head being at least partially guidable through the working opening for working on the jawbone;
a connection fluidly connected to the hollow body for establishing an internal pressure in the hollow; and
an adjustment means or adjustment mechanism provided in or on the device for axial movement or linear forward feed of the shaft in the hollow space along a longitudinal axis of the shaft and the hollow space.

18. The device of claim 17, wherein the hollow body is constructed as a tubular body.

19. The device of claim 17, wherein the working tool is an angular connector with an angular connector head.

20. The device of claim 17, wherein the working tool and the hollow body are interconnected with a constant predetermined distance to each other, preferably fixed in position to each other, and only the shaft is linearly movable relative to the working tool along the longitudinal axis of the shaft and the hollow space wherein the adjustment mechanism is preferably exclusively arranged in the working tool, preferably in an interior of the angular connector head.

21. The device of claim 18, wherein the working tool and the hollow body are connected with each other via a detachable connection.

22. The device of claim 21, wherein the detachable connection is a screw connection or a bayonet connection.

23. The device of claim 17, wherein the shaft is arranged at a fixed distance to the working tool or the angular connector head or is immovable relative to the working tool or the angular connector head in linear direction along the longitudinal axis of the hollow space, and wherein the working tool and the hollow body are connected with each other so that a distance between the working tool and the hollow body is adjustable, wherein the working tool and the hollow body are movable relative to each other exclusively linearly along the longitudinal axis of the shaft and the hollow space, or wherein the distance between the working tool and the hollow body is adjustable exclusively linearly along the longitudinal axis of the hollow space.

24. The device of claim 23, wherein the working tool and the hollow body are connected to each other via a straight threaded bolt, and wherein rotation of the threaded bolt causes a change of the distance between the working tool and the hollow body.

25. The device of claim 24, further comprising an external drive for effecting the rotation of the threaded bolt.

26. The device of claim 24, wherein the threaded bolt is oriented parallel to the longitudinal axis of the shaft and the hollow space.

27. The device of claim 23, further comprising at least one, optionally two guide pins protruding from the hollow body or from projections extending from the hollow body, said guide pins being oriented parallel to the longitudinal axis of the shaft and the hollow space and extending through and being guided in a recess of the working tool.

28. The device of claim 23, further comprising a spacer arranged between the working tool and the hollow body, wherein a volume and/or a thickness or height of the spacer is adjustable.

29. The device of to claim 28, wherein the spacer is constructed as a hollow body, and wherein the volume changes by introducing a fluid into the spacer or removing the fluid from the spacer.

30. The device of to claim 28, wherein the spacer is a balloon body which can be increased in size by introducing water into the balloon body.

31. The device of claim 28, wherein the spacer is a ring-shaped tire which surrounds and rests against the working tool and the hollow body in a region of the inlet opening.

32. The device of claim 17, wherein the working tool is an angular connector with a shaft having a longitudinal axis and extending from the angular connector head of the angular connector and rotating about the longitudinal axis.

33. The device of claim 32, wherein the tool head is configured as a milling cutter or drill head.

34. The device of claim 17, further comprising a single handle for simultaneously grasping, holding and operating the working tool and the hollow body.

35. The device of claim 17, wherein a distance by which the shaft is movable is maximally 1 cm, preferably about 0.56 cm.

36. The device of claim 17, wherein the inlet opening is closed with a sealing element which permits at least a linear adjustability of the shaft.

37. The device of claim 17, further comprising a guide element which is insertable with a precision fit into the inlet opening, thereby sealing the inlet opening pressure tight and essentially fluid tight, said guide element having a continuous recess through which the shaft is guidable and insertable into the hollow body, wherein the guide element optionally has a connection for introducing a working medium through the guide element into the hollow body for establishing an internal pressure in the hollow body, and wherein the guide element is optionally detachably fastenable to the hollow body and removable from the hollow body in a destruction free manner.

38. The device of claim 17, wherein the shaft is guided and supported, in particular in the recess in a pressure tight and essentially fluid tight manner so as to enable establishment of a pressure of at least 1.5 bar, preferably at least 2.5 bar in the interior of the hollow body and simultaneously enable at least a forward feed, a drive movement and/or control movement of the shaft, in particular a rotation, a circular or tumbling movement and/or an axial forward feed of the shaft.

39. The device of claim 17, wherein the rotation of the shaft is coupled with the linear forward feed of the shaft via a common drive.
